(19) United States
(12) Patent Application Publication
(10) Pub. No.: US 2025/0325606 A1
(43) Pub. Date: Oct. 23, 2025

(54) LACTICASEIBACILLUS PARACASEI DG(R) CNCM I-1572 DSM 34154 FOR USE IN A METHOD OF TREATING DYSBIOSIS IN PATIENTS WITH IBS

(71) Applicant: ALFASIGMA S.P.A., Bologna (IT)

(72) Inventors: Andrea Biffi, Bologna (IT); Simone Domenico GUGLIELMETTI, Bologna (IT); Walter Fiore, Bologna (IT)

(73) Assignee: ALFASIGMA S.P.A., Bologna (IT)

(21) Appl. No.: 18/865,784

(22) PCT Filed: May 17, 2023
(86) PCT No.: PCT/IB2023/055083

§ 371 Date: Nov. 14, 2024

(30) Foreign Application Priority Data
May 17, 2022 (IT) 102022000010244

(51) Int. Cl. A61K 35/747; A61P 1/00

(52) U.S. Cl. CPC A61K 35/747; A61P 1/00

(57) ABSTRACT

The object of the present invention is to use the strain of bacteria named Lacticaseibacillus paracasei DG® CNCM I-1572 DSM 34154 for use in a method of treating dysbiosis, particularly in patients with IBS (Irritable Bowel Syndrome), preferably without constipation, who have high fecal levels of Collinsella aerofaciens.

The object of the present invention is to use the strain of bacteria named Lacticaseibacillus paracasei DG® CNCM I-1572 DSM 34154 for use in a method of treating dysbiosis, particularly in patients with IBS (Irritable Bowel Syndrome), preferably without constipation, who have high fecal levels of Collinsella aerofaciens.

### TECHNICAL BACKGROUND

Irritable Bowel Syndrome (IBS) is one of the most common gastrointestinal disorders, affecting about 11% of the population in the United States and Europe, and in which recurrent abdominal pain is associated with changes in bowel habits. IBS has traditionally been viewed as a disorder possessing a psychosomatic component associated with bowel motor abnormalities and visceral hyperalgesia. Despite the lack of obvious physiologic irregularities at the level of the digestive tract, the use of quantitative morphologic and molecular techniques has made it possible to show, in a large percentage of patients with IBS, some alterations of the gastrointestinal mucosa and/or lumen at the tissue, cellular, and molecular levels.

There are also some experimental evidences suggesting a contribution of the qualitative/quantitative composition of the gut microbiota in the pathophysiology of IBS. Firstly, prospective studies have shown that 3% to 36% of enteric infections, inducing a marked disruption of the intestinal microbial ecosystem, lead, as a consequence, to a new diagnosis of IBS, termed post-infectious. Secondly, antibodies to flagellin (a globular protein present in the flagella of indigenous bacteria inhabiting the human gut), toxin B and vinculin (associated with the altered gut microbiota) as well as increased levels of human beta-defensin-2 (an antimicrobial inducible protein) have been identified in at least one subgroup of patients with IBS. These data suggest the presence of a host immune response to components of the gut microbiota. In addition, numerous studies report the presence of changes in the composition and stability over time of the gut microbiota in subjects with IBS. Although these studies are not fully comprehensive and cannot reach definitive conclusions, they still showed that the gut microbiota of patients with IBS differed significantly from controls.

Further evidence shows that the composition of the gut microbiota may somehow influence the pathophysiology of IBS syndrome is related to the fact that modulation of the gut microbiota with probiotics and nonabsorbable antibiotics is able, in some cases, to improve symptoms in patients, thus indicating that there is an implication of interactions between gut bacteria and host in the evolution and generation of symptoms in patients with IBS.

Probiotics are defined as live microorganisms that, when administered in adequate amounts, confer a health benefit to the host. Their functions include, for example, the prevention of overgrowth of potentially harmful bacteria in the intestine, the ability to increase the resistance of this anatomical district against invasion by pathogens, the induction of secretion of soluble factors, such as cytokines and antimicrobial peptides, as well as the strengthening of epithelial barrier function.

Reviews and meta-analyses of the scientific literature indicate that, in general, microorganisms belonging to the probiotic class can provide therapeutic benefit with respect to IBS symptoms. However, it was only when data from different studies were pooled, regardless of the probiotic used, that a general beneficial effect on symptoms was observed. In contrast, analyses of individual subgroups of patients responding to a particular treatment according to the type of probiotic used showed no statistically significant benefit of administration. The wide variety of species, strains, and doses of probiotics used in the different studies, sometimes done even on very limited case series, makes it difficult to come to specific conclusions about what, if any, the optimal probiotic strategy to use in the treatment of IBS might be.

Moreover, the mechanism of action by which probiotics exert their beneficial action in humans is largely incomplete; the few clinical studies available report that some strains possess anti-inflammatory properties and/or are able to act on the qualitative/quantitative composition of the microbiota. Indeed, there are specific bacterial strains that, more than others, are indicative of elevated levels of dysbiosis and are present in higher concentrations in IBS patients, including Ruminococcus bromii and Ruminococcus spp.

Among clinical studies, O'Mahony L. et al. (Gastroenterology 2005, 128) reports how Bifidobacterium longum subsp. infantis 35624, but not Lactobacillus salivarius, has been recognized to normalize the ratio of IL-10/IL-12 interleukins, indicative of a pro-inflammatory T-helper 1-type immune response, in patients with IBS. In a study of healthy volunteers, Ferrario C. et al. (J Nutr 2014, 144) report that intake of L. casei DG® (CNCM I-1572) can result in significant modulation of fecal levels of bacteria of the order Clostridiales (now re-designated Eubacteriales) and butyrate, with potential health benefits for the host. In addition, D'Inca et al. (Dig Dis Sci 2011, 56) demonstrated how rectal administration of L. casei DG® (CNCM I-1572) significantly reduced the levels of mRNAs encoding for TLR-4 and IL-1β, and significantly increased IL-10, in the colonic mucosa of patients with mild left-sided ulcerative colitis.

Khlinov et al. (Experimental & clinical gastroenterology, vol. 1 (6), 2021 Aug. 31, pages 57-62) reports a study on patients with IBS-C treated with mebeverine hydrochloride and L. paracasei DG with fructo-oligosaccharides versus placebo.

It is noted that the bacterial strain L. casei DG® (CNCM I-1572) or L. paracasei DG® (CNCM I-1572) was redeposited on Feb. 2, 2022 as Lacticaseibacillus paracasei DG I-1572 DSM 34154 following the reclassification of the genus Lactobacillus published by Zheng et al. in the scientific journal Int. J. Syst. Evol. Microbiol., 70(4):2782-2858, 2020. The above two designations are interchangeable with each other because they always refer to the same strain of bacteria.

The study by Cremon C. et al. (UEG Journal, 2018, 6) reports on a randomized pilot clinical trial investigating the effects of Lacticaseibacillus paracasei DG I-1572 DSM 34154 on clinical factors and microbiota composition in IBS patients with both diarrheal, constipated, mixed, and indefinite alvus. Specifically, administration of this strain resulted in a significant reduction in the bacterial genus Ruminococcus, an increase in short-chain fatty acids acetate and butyrate, and a reduction in IL-15. However, a statistically significant reduction in IBS symptoms could not be demonstrated.

Therefore, there is still a need for specific probiotic therapies that can act at the level of the gut microbiota and/or inflammation factors in patients with IBS and alleviate their symptoms, which are effective and safe to use and can overcome the disadvantages associated with known treatments.

### SUMMARY

It is an object of the present invention to provide a probiotic for use in a method of treating dysbiosis of IBS patients, preferably non-constipated IBS patients particularly to decrease or reduce the presence of Collinsella aerofaciens in the intestinal microbiota of said patients. Reducing dysbiosis also refers to reducing the abundance of intestinal pathobiont bacteria such as, for example, Bacteroides plebeius, Dorea spp., Ruminococcus bromii, Ruminococcus spp., Blautia spp. and Collinsella aerofaciens.

It is another purpose of the present invention to provide a probiotic to relieve intestinal symptoms effectively and safely in IBS patients, preferably in non-constipated IBS patients.

These and other purposes are achieved by the object of the present invention, which provides an effective probiotic for the treatment of dysbiosis of the intestinal microbiota of patients with IBS and the consequent decrease in the symptoms of said disease.

### BRIEF DESCRIPTION OF THE FIGURES

FIG. 1A: Study design for a total treatment duration of 18 weeks.

FIG. 1B: Study scheme (A) and summary of the IBS patients that concluded the PROBE-IBS/2 trial per protocol (PP) in the probiotic arm included in this study (B). NRS, numeric rating scale.

FIG. 2: Faecal bacterial taxa distinguishing responder (R) from non-responder (NR) non-constipated IBS patients. P values are according to Mann-Whitney test carried out with CLR-transformed bacterial abundances. P values with a red background indicate taxa increased in the R group. The heatmap represents the mean CLR-transformed abundances of the reported taxonomic units. The taxonomic lineage of each taxon is shown: p, phylum; c, class; o, order; f, family; g, genus; s, species. Corrections/updates of the taxonomy with respect to the nomenclature in the GreenGenes database are indicated in violet and were based on the NCBI Taxonomy database. Taxonomic names written in blue were determined through a manual BLASTN search in GenBank using the sequence of the corresponding reads. The histogram with black and green bars on the right indicates the contribution of each single bacterial taxon to the first two components of the PLS discriminant analysis (PLSDA) biplot represented in FIG. 3. Roman numerals are indicated to link bacterial taxa to the PLSDA loading plot of FIG. 3. The 11 bacterial taxa that significantly contributed to variability explanation in the PLSDA analysis are shown in bold.

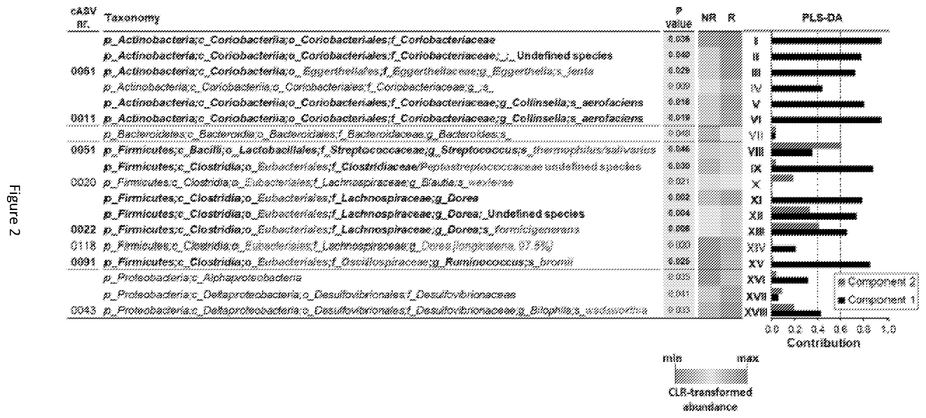

FIG. 3: Biplot of PLS discriminant analysis (PLSDA) with prediction background for responder (R) and non-responder (NR) non-constipated IBS patients (panel A) and for R, NR and healthy controls (HC) (panel B). Roman numerals in panel A refer to bacterial taxa in FIG. 2. The percentages indicate the explained variation at each axis.

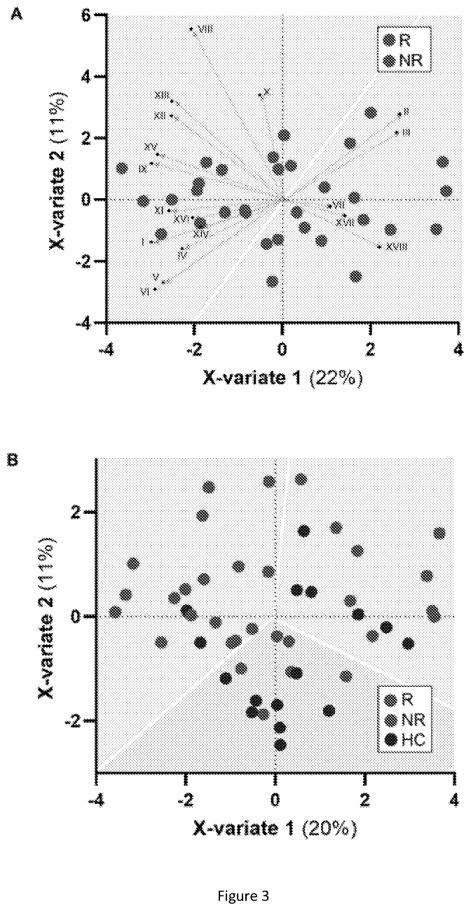

FIG. 4: Dot plot of the most abundant bacterial taxa found to better discriminate between responders and non-responder patients. HC, healthy controls (n=100); R and NR, responder and non-responder NC-IBS patients; other NC-IBS, other non-constipated IBS patients recruited at baseline (n=161); Statistics is according to Mann-Whitney test; *, P<0.05; **, P<0.01.

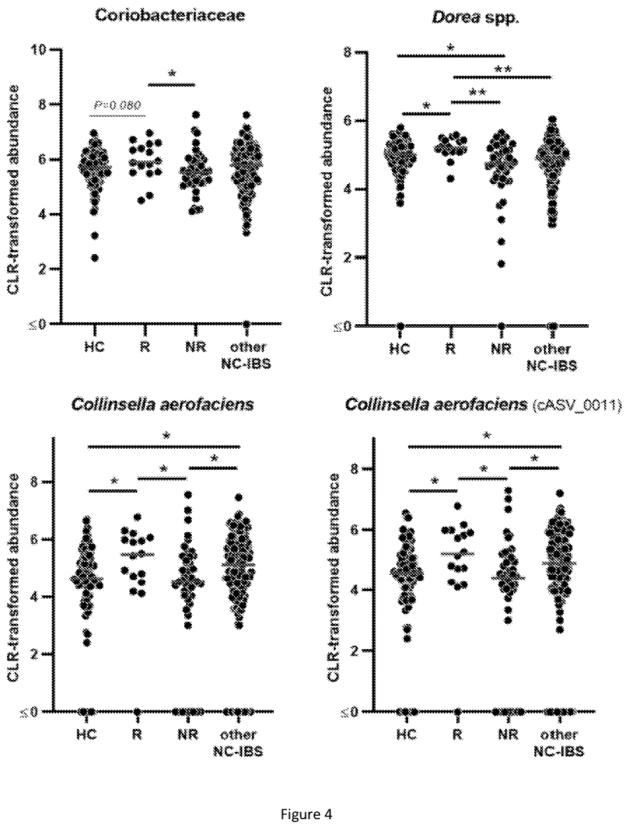

FIG. 5: Graphics of Linear discriminant analysis (LDA) effect size (LEfSe) for responder (R) NC-IBS patients in the probiotic arm of the trial. LDA scores indicate taxa significantly (P<0.05) higher before (V2; negative LDA) or after (V4; positive LDA) the probiotic intake. The name of the taxon levels is abbreviated as follows: p, phylum; c, class; o, order; f, family; g, genus; s, species.

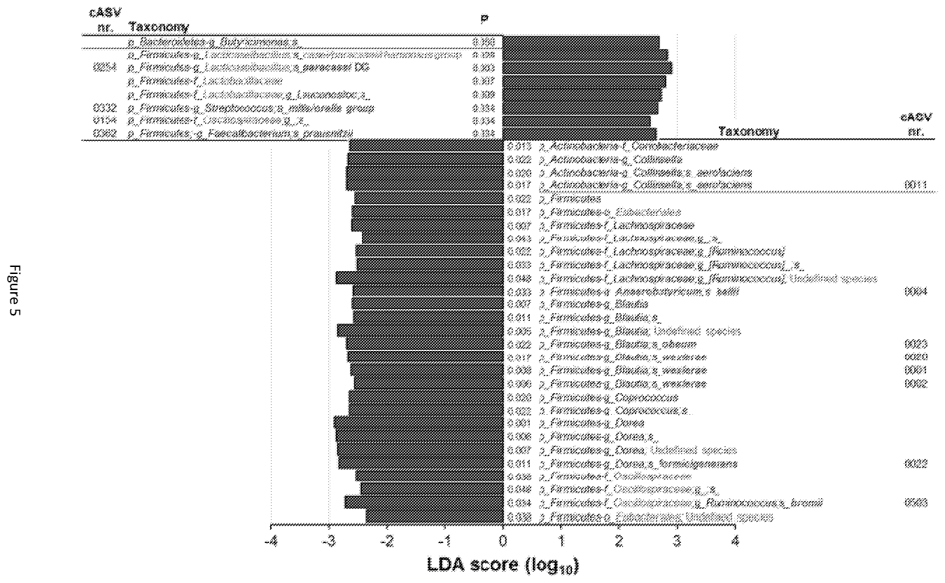

FIG. 6: Correlation analysis of bacterial taxa with serum markers for permeability (panel A), and liver and kidney functionality (panel B). In panel A, V1/V2 indicates correlation analysis performed with data from blood samples collected before run-in (visit V1) and faecal samples collected before the probiotic intervention (visit V2); V4 indicates correlation analysis performed with data from blood and faecal samples collected at end of the probiotic intervention (visit V4). Only taxa with the same significant correlations at V1/V2 and V4 are shown. The heatmap represents the τ coefficient of Kendall rank correlation. Asterisks indicate significance in the Kendall rank correlation: *P<0.05; **P<0.01; ***P<0.001. The taxonomic lineage of each taxon is shown: p, phylum; c, class; o, order; f, family; g, genus; s, species. ALT, alanine aminotransferase; AST, aspartate-aminotransferase; Bil, bilirubin; ALK, alkaline phosphatase; BUN, blood urea nitrogen; Crea, creatinine.

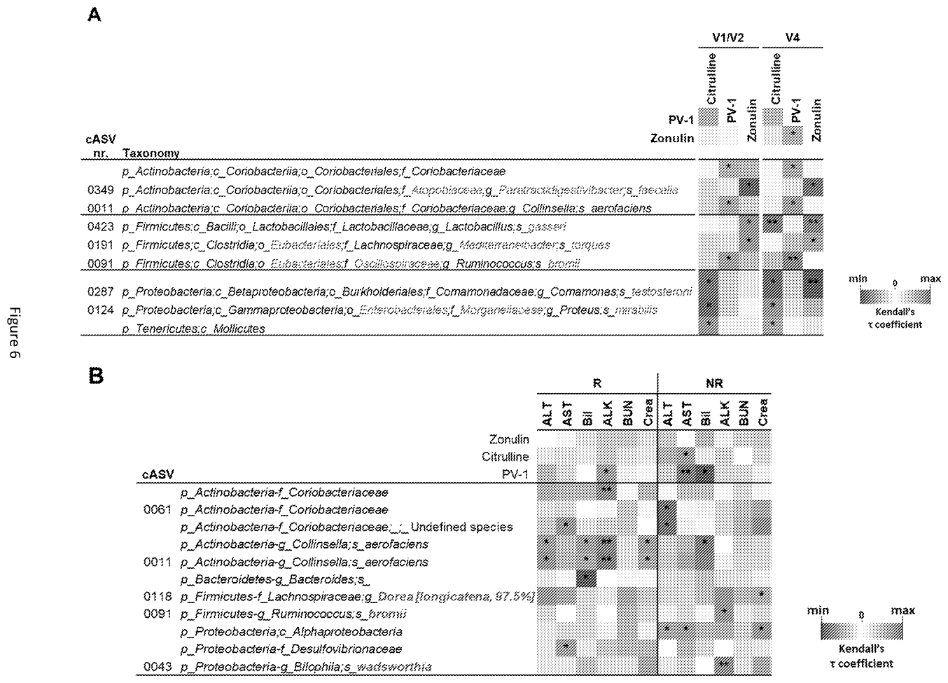

### DESCRIPTION OF THE INVENTION

According to one of its aspects, the present invention relates to a bacteria strain belonging to the species Lacticaseibacillus paracasei deposited as Lacticaseibacillus paracasei DG I-1572 DSM 34154 for use in a method of treating dysbiosis of the intestinal microbiota of subjects with IBS, wherein said subjects are classified as non-constipated.

Preferably, said subjects with IBS have elevated levels of Collinsella aerofaciens, Bacteroides plebeius, Dorea spp., Ruminococcus bromii, Ruminococcus spp., Blautia spp.

Preferably, said elevated levels of Collinsella aerofaciens, Bacteroides plebeius, Dorea spp., Ruminococcus bromii, Ruminococcus spp., Blautia spp. are present in the feces of subjects with IBS in need.

Preferably, said subjects are IBS sufferers, preferably they are non-constipated IBS sufferers.

Preferably, said bacteria it is administered orally, preferably in the form human supplement Enterolactis® Plus capsules.

Preferably, said human supplement is preferably administered twice daily, for a period of time preferably from 4 to 24 weeks, more preferably from 8 to 12 weeks.

Preferably, each of said capsules contains from 1×⁶ a 1×10¹², more preferably from 1×10⁸ to 1×10¹⁰, even more preferably from 10×10⁹ to 50×10⁹ CFU/capsule.

Preferably, said bacteria is present in each of said capsules in solid form, preferably powder, dry or lyophilized.

Preferably, the daily dose may comprise from 1 to 4 capsules, preferably from 2 to 3 capsules/day.

Preferably, said bacteria is for use in a method for the treatment of abdominal symptoms of IBS patients, preferably in non-constipated IBS patients.

According to one of its aspects, the invention relates to a probiotic based on Lacticaseibacillus paracasei DG I-1572 DSM 34154 for use in a method of treating dysbiosis of the intestinal microbiota of patients with IBS, especially in cases where said patients are classified as non-constipated.

According to one of its aspects, the invention relates to a probiotic based on Lacticaseibacillus paracasei DG® I-1572 DSM 34154 for use in a method of treating high levels of Collinsella aerofaciens, Bacteroides plebeius, Dorea spp., Ruminococcus bromii, Ruminococcus spp., Blautia spp. in the feces of subjects. Preferably, said Lacticaseibacillus paracasei DG® I-1572 DSM 34154 is for use in subjects who are IBS sufferers, preferably are non-constipated IBS sufferers. Preferably, said Lacticaseibacillus paracasei DG® I-1572 DSM 34154 is administered orally, preferably in the form Enterolactis® Plus capsules. Preferably, said probiotic Lacticaseibacillus paracasei DG® I-1572 DSM 34154 is administered preferably 2 times a day, for a period of time preferably from 4 to 24 weeks. Preferably, said Lacticaseibacillus paracasei DG® I-1572 DSM 34154 for use in the treatment of abdominal symptoms of IBS patients, preferably of non-constipated IBS patients.

In particular, Lacticaseibacillus paracasei DG® I-1572 DSM 34154 induces a decrease of abdominal pain in NC-IBS patients from 40% to 60%. It also improves the faecal type of the same patients.

In this description, dysbiosis of the intestinal microbiota is intended to identify a condition of alteration in the amount and type of microorganisms present in the gastro-intestinal tract compared with normal physiological conditions, which alterations are causally related to the pathological or dysfunctional condition.

The population of IBS patients considered non-constipated consists of IBS-D patients, with predominant diarrheal episodes, and IBS-M patients, with mixed bowel habits.

Lacticaseibacillus paracasei DG I-1572 DSM 34154 is present in a currently registered dietary supplement as Enterolactis® Plus and is also currently known by the registered trademark L. casei DG® (CNCM I-1572) or L. paracasei DG® (CNCM I-1572). It should be noted that the strain of bacteria L. casei DG® (CNCM I-1572) or L. paracasei DG® (CNCM I-1572) was redeposited on Feb. 2, 2022 as Lacticaseibacillus paracasei DG I-1572 DSM 34154 following the reclassification of the genus Lactobacillus published by Zheng et al. in the scientific journal Int. J. Syst. Evol. Microbiol., 70(4):2782-2858, 2020. The above two designations are interchangeable with each other because they always refer to the same strain of bacteria.

The probiotic for use according to the invention is employed by administering Enterolactis® Plus, capsules for oral use containing not less than 24 billion L. casei DG® (Lactobacillus paracasei CNCM I-1572) (Lacticaseibacillus paracasei DG I-1572 DSM 34154) per capsule, one to three capsules per day, preferably two capsules per day, for a period of time from 8 to 24 weeks, e.g., 12 weeks.

Preferably, said capsules are administered on an empty stomach, such as one hour before main meals, or even two hours after main meals.

As will be amply demonstrated in the experimental part that follows, the probiotic for use according to the invention is able to improve abdominal symptoms of non-constipated IBS patients. Said improvement is related to the ability of the probiotic for use according to the invention to intervene in the intestinal dysbiosis afflicting a specific subgroup of non-constipated IBS patients, and in particular to be able to decrease the presence of Collinsella aerofaciens in the intestinal microbiota of said patients.

As previously reported, the probiotic for use according to the invention, is Lacticaseibacillus paracasei DG I-1572 DSM 34154) which is administered orally and has been shown to be effective in the treatment of intestinal dysbiosis of non-constipated IBS patients, and in particular in cases of the presence of high levels of Collinsella aerofaciens in the intestinal microbiota.

According to another of its aspects, the invention has as its object a method for decreasing or reducing abdominal symptoms in subjects with IBS, which includes administering to said subjects who require it, one or more capsules, e.g., two capsules per day, of Enterolactis® Plus for use according to the invention, preferably for a period of time from 4 to 24 weeks, even more preferably from 8 to 12 weeks. Subject is understood here to mean a human being.

The probiotic for use according to the invention may be used alone or, if desired or necessary, in combination with other substances, provided that such substances do not counteract or limit its effects.

The following experimental section will present the results of the clinical study concerning the probiotic for use according to the invention in which, said use is compared with that of a placebo.

### Experimental Section

Objective: One of the aims of the present study is to identify markers for the recognition of non-constipated (NC) IBS patients that may have significant clinical improvements upon treatment with the probiotic strain Lacticaseibacillus paracasei DG (in short LDG).

Design: Post-hoc analysis of samples collected during a multi-center, randomized, double-blind, parallel-group, placebo-controlled trial, in which NC-IBS patients were randomized to receive at least 24 billion CFU LDG or placebo capsules b.i.d. for 12 weeks. Primary clinical endpoint was the composite response based on improved abdominal pain and fecal type. Fecal microbiome and serum markers of intestinal such as for example, PV1, liver and kidney functionality were investigated.

Results: Responders (R) in the probiotic arm (25%) differed from non-responders (NR) based on the abundance of 18 bacterial taxa including the family Coriobacteriaceae, Dorea spp., and Collinsella aerofaciens, which were overrepresented in R patients. These taxa also distinguished R (but not NR) from healthy controls. The probiotic intervention significantly reduced the abundance of these bacteria in R but not in NR. Analogous results emerged for C. aerofaciens from the analysis of data of a previous trial performed on IBS with the same probiotic. Finally, C. aerofaciens positively correlated with PV-1 and markers of liver functionality.

Advantageously, the bacteria strain L. casei DG® (Lacticaseibacillus paracasei DG I-1572 DSM 34154)—Enterolactis® Plus, .is effective on NC-IBS patients with a greater abundance of potential pathobionts. Among these, C. aerofaciens emerges as a potential predictor of the efficacy of probiotics.

### Study Objectives

Primary objective: to evaluate the effect of L. casei DG® on abdominal symptoms in non-constipated patients with irritable bowel syndrome (IBS), patients with symptoms meeting Rome IV criteria for diagnosis of IBS without constipation i.e., patients with IBS-D and IBS-M.

Secondary objectives: to evaluate the following parameters:
the presence of IBS symptoms;
daily stool consistency;
patient's overall satisfaction;
overall quality of life;
psychological impairment;
the intake of rescue medications;
the composition of the gut microbiota and metabolic products;
intestinal permeability;
the recovery of L. casei DG® strain in the feces.

### Experimental Design

Multicenter, randomized, double-blind, parallel-group, placebo-controlled study.

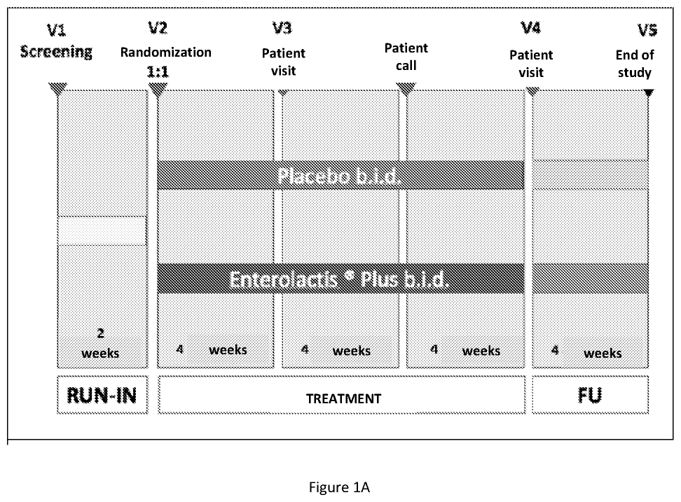

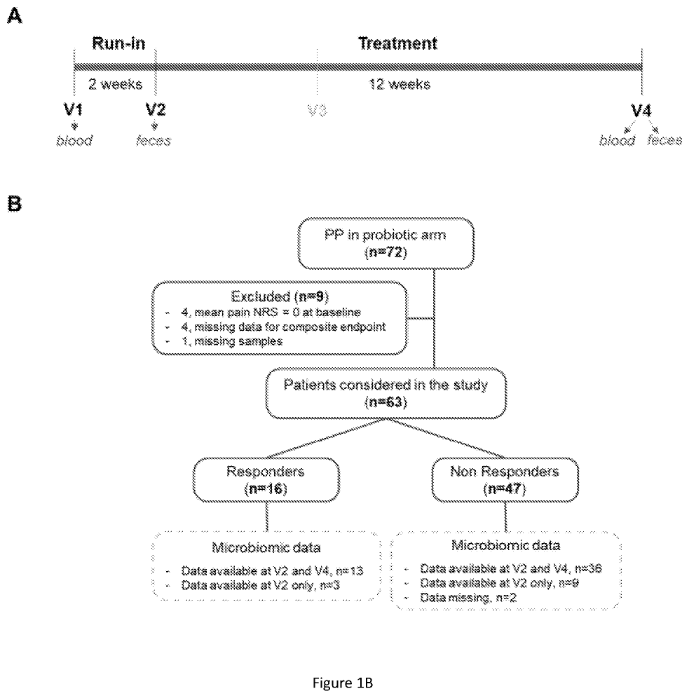

The study consisted of an initial (run-in) phase of 2 weeks during which data were collected on abdominal and alveus pain and discomfort, which were useful in establishing baseline levels used to evaluate treatment efficacy. This was followed by a 12-week phase (treatment) during which each patient took either two oral capsules per day of Enterolactis® Plus (a single-strain probiotic formulation containing at least 24 billion CFUs of L. paracasei DG) or two placebo capsules, which were physically indistinguishable from Enterolactis® Plus capsules. Finally, there followed 4 weeks in the early phase (follow-up) conditions, during which no capsules were taken. Each cycle thus had a total treatment duration of 18 weeks, as per the study design in FIGS. 1A and 1B.

Three fecal samples (during visits at the end of weeks 2, 14 and 18) and three blood samples (during the first visit at time zero and at visits at the end of weeks 14 and 18) were collected for each patient. These samples were subjected to analysis of various markers; in particular, taxonomic characterization of bacterial populations and quantification of short-chain fatty acids (acetate, butyrate, propionate, valerate, isovalerate, lactate, and succinate) were conducted on the feces, while the following were quantified in the blood samples: markers of permeability such as PV-1, liver [alanine aminotransferase (ALT), aspartate-aminotransferase (AST), bilirubin (Bil), alkaline phosphatase (ALK)] and kidney [blood urea nitrogen (BUN) and creatinine (Crea)] functionality.

### Patients

Patients selected for the present study were male or female and aged≥18 years, diagnosed with IBS without constipation according to Rome IV criteria.

The diagnostic criteria for IBS, which must be met for the 3 months preceding the study with onset of symptoms at least 6 months before diagnosis, consist of the presence of recurrent abdominal pain at least 1 day per week, associated with 2 or more of the following criteria:
related to defecation;
associated with change in stool frequency;
associated with change in the shape (appearance) of stools.

IBS patients without constipation include:
IBS with predominant diarrhea (IBS-D): more than a quarter (25%) of bowel movements with stool shape (Bristol) type 6 or 7;
IBS with mixed bowel habits (IBS-M): more than one quarter (25%) of bowel movements with stool shape (Bristol) type 1 or 2 and more than one quarter (25%) of bowel movements with stool shape (Bristol) type 6 or 7.

### Inclusion Criteria

age≥18 and ≤65 years;
positive diagnoses of IBS without constipation (IBS-D and IBS-M, both male and female) according to Rome IV ed. criteria;
negative result of a colonoscopy performed in the 5 years prior to the screening visit in case the patient is ≤50 years of age, or if the patient has any of these warning signs:
Has significant documented weight loss in the past 6 months; or
Has symptoms during the night; or
Has a family history of colon cancer; or
has blood mixed with the stool (excluding blood from hemorrhoids);
negative outcome for further relevant screening or consultations, where appropriate;
ability to adhere to the study protocol.

### Exclusion Criteria

patients with IBS-C or IBS-U in accordance with Rome IV ed. criteria;
presence of any relevant organic, systemic, or metabolic disease (particularly significant history of cardiac, renal, neurological, psychiatric, oncologic, endocrinologic, metabolic, or hepatic disease), or abnormal laboratory values, detected during the run-in period, considered clinically significant based on predefined values (e.g., renal or hepatic functional levels 2 times greater than upper reference values);
established organic bowel disease, including celiac disease, food allergies or inflammatory bowel disease (Crohn's disease, ulcerative colitis, diverticular disease, infectious colitis, ischemic colitis, microscopic colitis);
previous major abdominal surgery;
active malignancy of any kind, or history of malignancy (patients with a medical history of other malignancies that have been surgically removed and who have no evidence of recurrence for at least five years prior to study enrollment are acceptable);
untreated food intolerance as established or suspected lactose intolerance, as defined by medical history assessment or, if appropriate, lactose breath test;
use of probiotics or topical/systemic antibiotic therapy during the past month;
frequent or systematic use of contact laxatives;
pregnant women or women of reproductive age in the absence of effective contraceptive methods;
inability to adhere to the protocol;
treatment with any experimental drug within the previous 30 days;
recent history or suspected history of alcohol abuse or drug dependence;
presence of red or white flag according to Rome IV ed—Psychosocial Alarm Questionnaire for Functional Gastrointestinal Disorders criteria.

### Randomization

Enrollable patients enter a 2-week run-in phase and are then randomly assigned in a 1:1 ratio to Enterolactis® Plus treatment or the equivalent bacteria-free product (placebo), similar in color, texture and taste, to be taken twice daily for 12 weeks.

### Efficacy Assessment

Primary endpoint: Proportions of patients having a composite response over 12 weeks: patients recording on ≥50% of days a ≥30% reduction from their baseline mean score for their worst abdominal pain and, at the same time, a stool consistency≤5.

The standard 11-point numerical rating scale (0=none to 10=worst possible pain) will be used to measure abdominal pain, and for abnormal defecation, stool frequency and shape will be measured using the Bristol Stool Form Scale (BSFS).

### Secondary Endpoints

relief of IBS symptoms, which will be assessed as follows:
pain relief: ≥30% reduction from baseline in the score referring to the worst abdominal pain over a period of ≥50% days;
composite response (composite response) over each 4-week interval;
improvement in global symptom score: a score of 0 or 1, or ≥2 improvement from baseline over a period of days≥50%;
adequate relief of IBS symptoms over a period of ≥50% during the previous weeks (answer “yes” for ≥50% of the weeks to the following question, “In the last week, have you had adequate relief of IBS symptoms?”);
IBS-SSS score, assessed at time zero and at the end of treatment after 12 weeks (a reduction of at least 50 points is considered clinically significant);
improvement in stool consistency: stool consistency score≤5;
overall satisfaction with treatment assessed by VAS scale;
assessment of Quality of Life through validated Short-Form 12 Items Health Survey (SF-12), on a scale of 0 to 100;
intake of rescue medication;
gut microbiota composition and metabolic products (SCFA and free amino acids and biogenic amines);
intestinal permeability (using blood sample), with assessment of serum levels of zonulin, citrulline and PV-1;
recovery of L. casei DG® strain in feces, according to the method described in Arioli et al Front. Microbiol 2018. 9:1720.

### Safety Assessment

Vital parameters including blood pressure, heart rate and respiratory rate.
Physical examinations including assessment of hair and skin condition, lymph nodes, eyes, ENT, breast, respiratory, cardiovascular, abdomen, urogenital, pelvic, and rectal.
Laboratory tests:
Serum chemistry: glucose, blood urea nitrogen (BUN), aspartate aminotransferase (AST), alanine aminotransferase (ALT), bilirubin, alkaline phosphatase, creatinine;
hematology: complete count and blood formula, platelets.
Adverse events, monitored continuously during the study, and defined as the appearance of undesirable medical signs, symptoms, or conditions that occur after informed consent has been obtained from the patient.

### Discussion

A total of 72 patients completed the study in the probiotic group and 70 in the placebo group, compared with the total 300 initially recruited. The 300 patients recruited represent only the PP (per protocol) population and not the ITT (intention to treat) population that is usually considered in the primary analysis. Moreover, even within the PP population itself, not all PP patients (which would be about 235) were considered, but only those after elimination of patients with deviation. This choice was due to the fact that this way only those patients who had taken the probiotic product and completed the study and for whom it was possible to collect samples at the different visits were considered.

As previously anticipated, the primary end-point of the study was the percentage of patients who had, over the 12-week period of probiotic or placebo administration, a composite response (composite response), i.e., patients who had on greater than or equal to 50 percent of the days a ≥30 percent reduction from the baseline mean score for their worst abdominal pain and, on the same days, a stool consistency score<5.

In accordance with the primary endpoint, 16 patients in the probiotic group (23.5%) and 19 in the placebo group (29.2%) were found to respond positively to the probiotic for use according to the invention (R, responder).

Table 1 reports the abdominal pain and faecal type at visit 2 and visit 4 of patients who received the treatment.

TABLE 1

| | Total | Responders | Non-responders |
|---|---|---|---|
| | (n = 63; 100%) | (n = 16; 25.4%) | (n = 47; 74.6%) |
| Abdominal | 3.4 ± 1.8 | 3.0 ± 1.4 | 3.6 ± 1.9 |
| pain during | | | |
| 2-week run-in | | | |
| (mean NRS) | | | |
| Abdominal | 3.1 ± 1.7 | 1.7 ± 1.1*/+ | 3.5 ± 1.6 |
| pain during | | | |
| 14-week | | | |
| treatment | | | |
| (mean NRS) | | | |
| Faecal type | 4.6 ± 1.1 | 4.5 ± 1.8 | 4.7 ± 1.2 |
| during | | | |
| 2-week run-in | | | |
| (mean number) | | | |
| Faecal type | 4.5 ± 1.0 | 4.1 ± 1.6* | 4.6 ± 1.1 |
| during | | | |
| 14-week | | | |
| treatment | | | |
| (mean number) | | | |

*, significantly different (P < 0.001) compared to run-in (baseline) in the R group
+, significantly different (P < 0.0001) compared to the abdominal pain after the 14-week treatment in the NR group

An explanation of the results obtained follows.
(i) patients with no pain or a too low degree of pain at baseline (IBS-SSS total score≤175 and NRS≤3) to show significant post-treatment improvement were included;
(ii) the diary card reports the requirement to indicate the degree of severity of “pain/annoyance” on a daily basis (the ability to report symptom annoyance interchangeably with pain makes it unclear whether patients wanted to report one or the other of the symptoms and makes it uncertain whether the comparison is between pain/annoyance at baseline and/or pain/annoyance at follow-up visits);
(iii) analysis of the daily diary, completed by patients during the two-week run-in, found that many patients did not meet Rome IV criteria for the diagnosis of IBS-D or IBS-M, although adherence to the inclusion criteria was also confirmed at baseline;
(iv) this resulted in a decisive reduction of the valid sample for the purpose of statistical analysis of the results because 183 patients out of 264 in the ITT population were eliminated;
(v) in addition, regarding treatment adherence, the microbiota analysis showed that 25 patients in the Enterolactis arm were not found to have L. casei DG® (Lacticaseibacillus paracasei DG I-1572 DSM 34154) (Enterolactis® Plus) (this means that the product may not have been not taken—fully or, at any rate, correctly—by the patient), while in 5 patients in the placebo arm L. casei DG® was found (a component present in other products in the line freely available in pharmacies, which the patient may have been free to find and take).

Significant changes in the amount and type of bacterial populations in the microbiota of placebo-treated subjects were much smaller than those observed in the group of subjects taking Enterolactis® Plus, suggesting that the improvement found in R subjects in the placebo group may not be related to changes at the level of the microbiota, as is the case with Enterolactis® Plus-treated patients, but rather to psychological factors, the psychosomatic component of IBS being known.

In order to determine whether patients who benefited from probiotic treatment had characteristics that distinguished them from patients who did not experience significant improvement (R vs NR, Non Responder), the levels of bacteria and other markers were compared between the two groups of patients. This analysis showed that R patients were characterized by a significantly higher abundance of several microbial groups indicative of dysbiosis in IBS patients than in healthy subjects, such as: Bacteroides plebeius, Dorea spp., Ruminococcus bromii, Ruminococcus spp., Blautia spp. and Collinsella aerofaciens. Specifically, principal component analysis based on the abundances of the 46 bacterial taxa found to be significantly different between R and NR patients showed that the most important taxonomic group for distinguishing R from NR patients was the species Collinsella aerofaciens.

The same type of analysis was also conducted among R and NR patients in the placebo group; in this case, fewer significantly different taxa were found to be present. Notably, no significant difference was found for Collinsella aerofaciens between R and NR patients.

A subsequent statistical analysis showed that the probiotic contained in the Enterolactis® Plus formulation for use according to the invention was able to result in a reduction (trending toward significance, P=0.0681) of Collinsella aerofaciens in responder (R) patients. In contrast, the concentration of Collinsella aerofaciens was not changed by administration of the probiotic for use according to the invention in either NR patients or placebo group patients.

Further investigation verified how non-constipated IBS patients, and especially responder subjects to treatment with Enterolactis® Plus for use according to the invention, exhibit significantly higher initial fecal levels of Collinsella aerofaciens than a population consisting of 100 healthy adults (control).

In addition, it was shown that patients who showed, through the use of the probiotic for use according to the invention, a reduction in the levels of Collinsella aerofaciens, simultaneously manifested a significant decrease in abdominal pain (P=0.0497) and a trend of decreasing fecal type (P=0.0709), suggesting that said bacterial strain may be mechanistically related to IBS symptomatology.

In addition to demonstrating the efficacy of the probiotic for use according to the invention with respect to its ability to decrease the abdominal symptomatology of non-constipated IBS patients by inducing a reduction in the concentration of Collinsella aerofaciens, the experimental data from the described clinical protocol allow us to highlight the possibility of measuring the concentration of this microorganism as a predictive test with respect to whether or not a given patient will respond to the probiotic L. casei DG® for use according to the present invention.

### Organic Acids Quantification

Organic acids (acetate, butyrate, propionate, valerate, isovalerate, lactate, and succinate) were detected and quantified in fecal samples by Ultra-Performance Liquid Chromatography—High-Resolution Mass Spectrometry (UPLC-HR-MS) on Acquity UPLC separation module (Waters, Milford, MA) coupled with an Exactive Orbitrap MS through a HESI-II probe for electrospray ionization (Thermo Scientific, San Jose, CA), as previously described in Gargari G, et al. (Environ Microbiol 2018; 20:3201-13).

Table 2 reports the organic acids quantification measured at visit V2 and visit V4.

TABLE 2

| | | | | Non- |
|---|---|---|---|---|
| Faecal organic | | Total | Responders | responders |
| acids | Visit | (n = 51) | (n = 13) | (n = 38) |
| Acetate (mmol/100 | V2 | 3.4 ± 3.5 | 2.6 ± 1.7 | 3.6 ± 3.9 |
| g of feces) | V4 | 3.7 ± 3.1 | 3.0 ± 1.8 | 3.9 ± 3.4 |
| Butyrate (mmol/100 | V2 | 3.3 ± 2.9 | 3.5 ± 2.4 | 3.3 ± 3.1 |
| g of feces) | V4 | 3.7 ± 3.3 | 4.6 ± 3.5 | 3.5 ± 3.3 |
| Propionate (mmol/100 | V2 | 1.3 ± 1.1 | 1.4 ± 0.9 | 1.3 ± 1.1 |
| g of feces) | V4 | 1.5 ± 1.9 | 1.5 ± 1.0 | 1.5 ± 2.1 |
| Valerate (mmol/100 | V2 | 1.3 ± 1.2 | 1.4 ± 0.6 | 1.2 ± 1.3 |
| g of feces) | V4 | 1.4 ± 1.0 | 1.4 ± 1.0 | 1.3 ± 1.0 |
| Isovalerate (mmol/100 | V2 | 1.0 ± 0.8 | 0.9 ± 0.5 | 1.0 ± 0.9 |
| g of feces) | V4 | 1.0 ± 0.7 | 1.0 ± 0.8 | 1.0 ± 0.7 |
| Lactate (mmol/1 kg | V2 | 2.2 ± 14.1 | 0.05 ± 0.10 | 2.9 ± 16.3 |
| of feces) | V4 | 3.9 ± 24.4 | 0.2 ± 0.5 | 5.2 ± 28.2 |
| Succinate (mmol/1 kg | V2 | 2.3 ± 7.7 | 0.3 ± 0.7 | 3.0 ± 8.9 |
| of feces) | V4 | 5.5 ± 16.1 | 9.8 ± 26.0 | 4.0 ± 10.9 |

### PV-1 Quantification

The endothelial permeability marker plasmalemma vesicle associated protein (PLVAP)/PV-1 was measured in serum samples by means of the Human PVLAP ELISA Kit (Fine test, China). Samples were processed according to manufacturers' instruction and absorbance at 450 nm was measured using an Eon plate reader. Absorbance data were then interpolated by means of a logarithmic standard curve, which was calculated for each single plate analyzed.

TABLE 3

| | | Total | Responders | Non-responders |
|---|---|---|---|---|
| Serum markers | Visit | (n = 61/511) | (n = 16/141) | (n = 47/371) |
| PV-1 (ng/ml) | V1 | 3.6 ± 1.6 | 3.7 ± 1.8 | 3.5 ± 1.6 |
| | V4 | 4.7 ± 4.3 | 7.0 ± 6.7 | 3.8 ± 2.5 |

1, the first and second numbers indicate “n” at V1 and V4, respectively

### Analysis of Liver and Kidney Functionality Markers

The following markers of liver and kidney functionality were assessed in serum samples: alanine aminotransferase (ALT), aspartate-aminotransferase (AST), bilirubin (Bil), alkaline phosphatase (ALK), blood urea nitrogen (BUN), and creatinine (Crea). AST and ALT were assessed by enzymatic dosage without activation by pyridoxal phosphate (Cobas 8000—Roche Diagnostics). Bil and ALK were dosed by enzymatic colorimetric test (Cobas 8000—Roche Diagnostics). Kinetic enzymatic test was performed to evaluate urea, and creatinine was assessed by kinetic staining test (Jaffé method) (Cobas 8000—Roche Diagnostics).

TABLE 4

| | | | | Non- |
|---|---|---|---|---|
| | | Total | Responders | responders |
| Serum markers | Visit | (n = 61/511) | (n = 16/141) | (n = 47/371) |
| Alanine | V1 | 20.1 ± 9.9 | 18.1 ± 7.1 | 20.7 ± 10.6 |
| aminotransferase (U/I) | V4 | 20.8 ± 10.3 | 19.6 ± 9.4 | 21.2 ± 10.7 |
| Aspartate | V1 | 19.8 ± 5.7 | 19.7 ± 6.5 | 19.8 ± 5.5 |
| aminotransferase (U/I) | V4 | 20.0 ± 6.5 | 18.7 ± 4.5 | 20.5 ± 7.0 |
| Bilirubin (mg/dl) | V1 | 1.3 ± 3.1 | 1.0 ± 1.8 | 1.3 ± 3.4 |
| | V4 | 1.1 ± 2.2 | 0.9 ± 1.3 | 1.2 ± 2.5 |
| Alkaline phosphatase | V1 | 62.2 ± 17.8 | 64.3 ± 19.9 | 61.5 ± 17.2 |
| (U/I) | V4 | 61.8 ± 16.5 | 64.0 ± 19.3 | 61.1 ± 15.6 |
| Blood urea nitrogen | V1 | 25.4 ± 10.4 | 26.2 ± 10.4 | 25.1 ± 10.5 |
| (mg/dl) | V4 | 25.5 ± 12.0 | 25.0 ± 10.8 | 25.7 ± 12.5 |
| Creatinine (mg/dl) | V1 | 3.6 ± 15.8 | 5.7 ± 19.3 | 2.9 ± 14.6 |
| | V4 | 4.1 ± 16.9 | 6.6 ± 21.4 | 3.2 ± 15.2 |

1, the first and second numbers indicate “n” at V1 and V4, respectively

1. A method of treating dysbiosis of intestinal microbiota with subjects suffering from IBS, wherein said subjects are classified as non-constipated with a bacteria strain belonging to the species Lacticaseibacillus paracasei deposited as Lacticaseibacillus paracasei DG I-1572 DSM 34154, said method comprising
administering to said subjects a pharmaceutical effective amount to said bacteria strain.

2. The method according to claim 1, wherein said subjects with IBS have elevated levels of Collinsella aerofaciens, Bacteroides plebeius, Dorea spp., Ruminococcus bromii, Ruminococcus spp., Blautia spp.

3. The method according to claim 1 or 2, wherein said elevated levels of Collinsella aerofaciens, Bacteroides plebeius, Dorea spp., Ruminococcus bromii, Ruminococcus spp., Blautia spp. are present in the feces of subjects with IBS in need.

4. (canceled)

5. The method according to claim 1, wherein said bacteria is administered orally, preferably in the form human supplement Enterolactis® Plus capsules.

6. The method according to claim 16, wherein said human supplement is administered twice daily, for a period of time from 4 to 24 weeks.

7. The method according to claim 16, wherein each of said capsules contains from 1×⁶ a 1×10¹² CFU/capsule.

8. The method according to claim 16, wherein said bacteria is present in each of said capsules in solid form.

9. The method according to claim 6, wherein the daily dose comprises from 1 to 4 capsules/day.

10. The method according to claim 1, wherein said non-constipated subjects suffer abdominal symptoms of IBS.

11. The method according to claim 6, wherein said human supplement is administered twice a daily for a period of time from 8 to 12 weeks.

12. The method according to claim 7, wherein each of said capsules contains from 1×10⁸ to 1×10¹⁰ CFU/capsule.

13. The method according to claim 7, wherein each of said capsules contains from 10×10⁹ to 50×10⁹ CFU/capsule.

14. The method according to claim 8, wherein said bacteria is present in powder, dry or lyophilized form.

15. The method according to claim 9, wherein the daily dose comprises from 2 to 3 capsules/day.

16. The method according to claim 5, wherein said bacteria is administered as human supplement Enterolactis® Plus capsules.
